(19) United States
(12) Patent Application Publication
(10) Pub. No.: US 2005/0131418 A1
(43) Pub. Date: Jun. 16, 2005

(54) Intramedullary nail drill guide

(76) Inventor: Amit Mor, Rehovot (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 10/734,118

(22) Filed: Dec. 15, 2003

(51) Int. Cl. A61B 17/58

(52) U.S. Cl. 606/96

(57) ABSTRACT

An intramedullary nail drilling guide including an intramedullary nail drilling guide including a radiation device including a radiation source and a radiation receptor, the radiation source being operative to emit a radiation beam along a beam axis, and a drill guide attached to a portion of the radiation device, the drill guide having a drilling aperture alignable with the beam axis.

### FIELD OF THE INVENTION

The present invention relates generally to an intramedullary nail drill guide for guiding a drill bit to drill transverse holes of an intramedullary nail that has been implanted in a bone of a patient.

### BACKGROUND OF THE INVENTION

In repairing broken bones, and particularly long bones of the body such as the humerus, femur or tibia, a common surgical practice is to insert a part known as an intramedullary nail into the interior portion of the bone. The intramedullary nail is then secured in place with respect to the bone by installing one or more transversely-extending screws or bolts at the proximal and distal portions of the intramedullary nail (sometimes only at the distal portions). The intramedullary nail serves to enhance the healing of the broken bone by providing a rigid structure about which the broken portions of the bone may adhere.

In order to install the screws or bolts in the intramedullary nail and bone, a screw hole must be drilled transverse to the bone in direct alignment with each transverse hole in the intramedullary nail. This drilling is performed after the intramedullary nail has been inserted into the bone canal. It is desirable that drilling should pass cleanly through the transverse holes, to prevent metal shreds and shavings from being formed when the drill touches the nail. Such shavings may impede healing, and may cause post-operation discomfort or pain for the patient. Further, if the hole was not properly drilled, a second or even a third through hole may have to be drilled, weakening the bone and delaying the healing process.

A problem with this procedure is that it is not possible to see the transverse holes directly, since the intramedullary nail is disposed inside the bone canal. Many solutions have been proposed to this problem in the patent literature.

For example, PCT published application WO0230258 describes a targeting system that includes a first fixation system, a second fixation system, and an aiming guide. After implantation within the intramedullary canal of a bone, the distal screw holes of an intramedullary nail are first aligned using conventional radioscopy equipment. The targeting system is then introduced between the radioscopy equipment and the patient such that the aiming guide of the targeting system is roughly aligned with the distal screw holes. The first fixation system is adapted to be fixedly coupled to an operating surface upon which an intramedullary nail fixation procedure may be performed. The second fixation system is adapted to be fixedly coupled to the handle of the implanted intramedullary nail. Rough adjustments to the first and second fixation systems enable an initial positioning of a drill with a first distal screw hole. The aiming guide, which is disposed between and fixedly coupled to the first fixation system and the second fixation system, is used to perfect alignment of the drill with the first distal screw hole. Once a precise alignment with the first distal screw hole has been realized, a mechanical adjustment to the aiming guide may allow for a rapid alignment of the drill with the second distal screw hole of the implanted intramedullary nail.

In other words, the drill guide must be placed between the radiation source and the intramedullary nail. The surgeon must hold the guide with one hand and drill with the other. This may make use of the device difficult and cumbersome.

PCT published applications WO0189395 and WO03043508 both describe extensions on the intramedullary nail which act as drilling guides.

Japanese Patent Document JP2000312680 describes a hand-held laser beam drill guide for intramedullary nails. The transverse holes of the intramedullary nail are aligned with a laser beam emanating from a hand-held drill guide.

In none of the prior art is there a connection between the drill guide and the radiation device.

### SUMMARY OF THE INVENTION

The present invention seeks to provide a novel intramedullary nail drilling guide, as is described more in detail hereinbelow.

There is thus provided in accordance with an embodiment of the present invention an intramedullary nail drilling guide including an intramedullary nail drilling guide including a radiation device including a radiation source and a radiation receptor, the radiation source being operative to emit a radiation beam along a beam axis, and a drill guide attached to a portion of the radiation device, the drill guide having a drilling aperture alignable with the beam axis.

In accordance with an embodiment of the present invention the drill guide is adjustably mounted on a base, which is attached to a portion of the radiation device. The base may be adjustably attached to a portion of the radiation device.

Further in accordance with an embodiment of the present invention a drill is provided that is movable towards and away from the drilling aperture.

Still further in accordance with an embodiment of the present invention the drill is mounted on a guide rail.

In accordance with an embodiment of the present invention at least a portion of the drill guide is radiolucent or radio-opaque.

Further in accordance with an embodiment of the present invention a locking device is adapted to spatially fix the drill guide.

### BRIEF DESCRIPTION OF THE DRAWINGS

The present invention will be understood and appreciated more fully from the following detailed description taken in conjunction with the drawing in which:

FIG. 1 is a simplified illustration of an intramedullary nail drilling guide, constructed and operative in accordance with an embodiment of the present invention.

### DETAILED DESCRIPTION OF THE PREFERRED EMBODIMENT

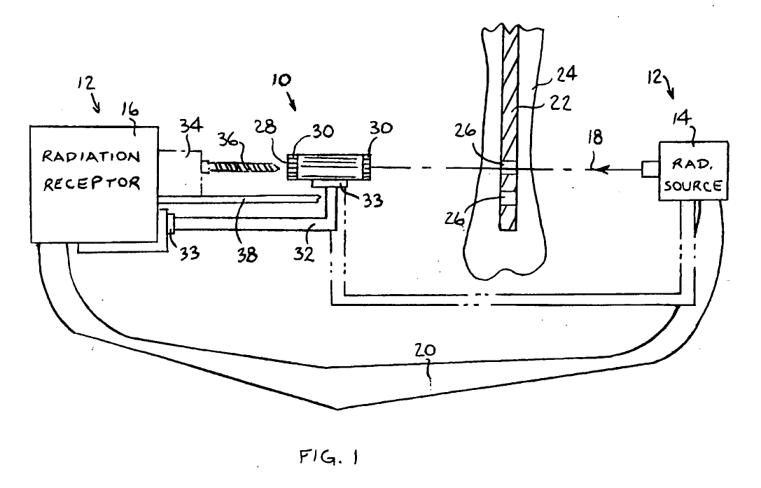

Reference is now made to FIG. 1. A radiation device 12 may be provided that includes a radiation source 14 (such as, but not limited to, an x-ray radiation source or ultrasound, for example) and a radiation receptor 16. Radiation source 14 may emit a radiation beam along a beam axis 18. Radiation source 14 and radiation receptor 16 may be mounted on a C-arm 20. Imaging equipment (not shown) may be provided for capturing and processing images obtained by the use of radiation device 12, as is well known in the art.

Radiation device 12 may provide images of an intramedullary nail 22 that has been placed into the interior portion of a bone 24 (e.g., a femur). Intramedullary nail 22 has a distal portion with one or more transverse holes 26. It is desired to drill holes into bone 24 that are aligned with transverse holes 26, so that one or more transversely extending screws or bolts may be installed through the drilled holes and through holes 26 to lock intramedullary nail 22 in place.

In accordance with an embodiment of the present invention, a drill guide 10 is attached to a portion of radiation device 12. For example, drill guide 10 may be attached, without limitation, to either one of or both radiation source 14 and radiation receptor 16, or to C-arm 20. Drill guide 10 may be of any size, shape or configuration, and may be constructed of any suitable material, such as but not limited to, metal, plastic ceramic, etc. In the non-limiting example illustrated in the drawing, drill guide 10 may have an elongate cylindrical body with a drilling aperture 28 alignable with the beam axis 18. Annular caps or rings 30 may be places at one or both ends of drill guide 10. Rings 30 may be radiolucent or radio-opaque, which may facilitate seeing them with the imaging equipment.

Drill guide 10 may be adjustably mounted on a base 32, which may be attached to some portion of radiation device 12 (e.g., either one of or both radiation source 14 and radiation receptor 16). “Adjustable mounted” means that drill guide 10 may be translated in any linear direction (e.g., up/down, sideways, back/forth, etc.) and/or any rotary motion (e.g., azimuth, elevation, roll, etc.) with respect to base 32. Additionally or alternatively, base 32 may be adjustably attached to radiation device 12. Base 32 may be rigid or flexible, and may be accordingly constructed of any suitable material. A locking device 33, such as but not limited to, a thumbscrew, clamp, etc., may be provided for spatially fixing drill guide 10 in place during drilling. Likewise, another locking device 33 may lock base 32 in place.

A drill 34 with a drill bit 36 may be provided for drilling the holes in the bone 24 through drill guide 10. Drill 34 may be mounted on a guide rail 38 and may be moved towards and away from drilling aperture 28.

In a typical use of drill guide 10, radiation device 12 is used to locate transverse holes 26. During location of holes 26, drill guide 10 may be moved out of the way of beam axis 18 so as not to interfere with sighting the holes 26. Drill guide 10 may then be moved so that the drilling apertures 28 are aligned with the holes 26 as seen in the imaging equipment used with radiation device 12. Drill guide 10 may then be locked in place with locking device 33. Drill 34 may then drill holes in bone 24 with drill bit 36 passing through and being guided by drilling apertures 28 of drill guide 10. The holes drilled in this manner are aligned with transverse holes 26, so that one or more transversely extending screws or bolts (not shown) may be installed through the drilled holes and through holes 26 to lock intramedullary nail 22 in place.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

1. An intramedullary nail drilling guide comprising:
a radiation device comprising a radiation source and a radiation receptor, said radiation source being operative to emit a radiation beam along a beam axis; and
a drill guide attached to a portion of said radiation device, said drill guide having a drilling aperture alignable with the beam axis.

2. The intramedullary nail drilling guide according to claim 1, wherein said drill guide is adjustably mounted on a base which is attached to a portion of said radiation device.

3. The intramedullary nail drilling guide according to claim 2, wherein said base is adjustably attached to a portion of said radiation device.

4. The intramedullary nail drilling guide according to claim 1, further comprising a drill movable towards and away from said drilling aperture.

5. The intramedullary nail drilling guide according to claim 4, wherein said drill is mounted on a guide rail.

6. The intramedullary nail drilling guide according to claim 1, wherein at least a portion of said drill guide is radiolucent.

7. The intramedullary nail drilling guide according to claim 1, wherein at least a portion of said drill guide is radio-opaque.

8. The intramedullary nail drilling guide according to claim 1, further comprising a locking device adapted to spatially fix said drill guide.

9. The intramedullary nail drilling guide according to claim 1, wherein said radiation source and said radiation receptor are mounted on a C-arm, and said drill guide is attached to said C-arm.
